(19) United States
(12) Patent Application Publication
(10) Pub. No.: US 2021/0282720 A1
(43) Pub. Date: Sep. 16, 2021

(54) METHOD OF ESTABLISHING BLOOD PRESSURE MODEL

(71) Applicants: Inventec (Pudong) Technology Corporation, Shanghai (CN); INVENTEC CORPORATION, Taipei (TW)

(72) Inventors: JONATHAN HANS SOESENO, Taipei (TW); Trista Pei-Chun CHEN, Taipei (TW); Wei-Chao CHEN, Taipei (TW)

(21) Appl. No.: 16/849,173

(22) Filed: Apr. 15, 2020

(30) Foreign Application Priority Data
Mar. 15, 2020 (CN) 202010178888.3

(51) Int. Cl. A61B 5/00; A61B 5/021; A61B 5/024; A61B 5/0402; G16H 50/50; G16H 50/30; G06N 3/08

(52) U.S. Cl. CPC A61B 5/7278; A61B 5/02125; A61B 5/02416; A61B 5/0402; G06N 3/08; A61B 5/725; G16H 50/50; G16H 50/30; A61B 5/7267

(57) ABSTRACT

A method of establishing blood pressure model comprising: obtaining a plurality of first physiologic data of a plurality of general users and a plurality of first blood pressure data of the plurality of general users; performing a deep learning algorithm to establish a general blood pressure model according to the plurality of first physiologic data and the plurality of first blood pressure data, wherein the general blood pressure model has a parameter set and a loss function; obtaining a second physiologic data of a specific user and a second blood pressure data of the specific user; generating a blood pressure estimation according to the second physiologic data and the parameter set; calculating an error according to the blood pressure estimation, the second blood pressure data and the loss function; and adjusting the parameter set to establish a specific blood pressure model according to the error.

### CROSS-REFERENCE TO RELATED APPLICATIONS

This non-provisional application claims priority under 35 U. S. C. § 119(a) on Patent Application No(s). 202010178888.3 filed in China on Mar. 15, 2020, the entire contents of which are hereby incorporated by reference.

### BACKGROUND

### 1. Technical Field

This disclosure relates to the blood pressure measurement, and more particularly to the blood pressure measurement system and method thereof based on the electrocardiography (ECG), the photoplethysmography (PPG) and the pulse transit time (PTT).

### 2. Related Art

Cardiovascular related diseases have been proven to be highly related to heart rate and blood pressure. Uncontrolled high blood pressure (BP) can lead to heart attack, stroke, heart failure, and other serious life threats. Therefore, accurate measurement of blood pressure is necessary to prevent unwanted events. According to the validation protocol followed by American National Standards Institute (ANSI), Association for the Advancement of Medical Instrumentation (AAMI) and International Organization for Standardization (ISO) in 2018, the tolerable error of blood measurement is equal to or less than 10 millimeters of mercury (mm Hg) with an estimated probability of 85% at least.

The blood pressure measurement method can be separated into two categories, namely, the cuff-based method and the cuffless method. The cuff-based method is intrusive because one of the user's arm has to be cuffed for at least 30 seconds to obtain an accurate reading. Therefore, cuff-based method is not suitable for blood pressure measurement for a long time (for example, all day). However, the cuff-based method can accurately measure a user's blood pressure. On the other hand, the cuffless method relies on sensors that are attached to the user's body, the sensors are used for obtaining sensed data of the user, such as one of the user's ECG, PPG, and PTT data, and then the sensing data is converted into a blood pressure value. Since the volume of the sensor is smaller than that of the cuff, the cuffless method is less intrusive and thus can continuously measure blood pressures all day long. However, since the result of the cuff-based blood pressure measurement is considered “gold standard”, the cuffless blood pressure measurement is naturally less accurate. In addition, the cuffless method needs to collect a plurality of sensing data of the user in a variety of situations such as walking, sitting, taking exercises in order to provide a relatively accurate blood pressure measurement. Therefore, the user needs to take an extra effort and time to provide sensing data in different situations.

### SUMMARY

In view of the above, the present disclosure proposes a method of establishing a specific blood pressure model. On the premise of preserving the advantage of the cuffless sphygmomanometer that can be worn on the body and can continuously measure, the accuracy of blood pressure measurement is improved, and the user is less interfered.

According to one or more embodiment of this disclosure, a method of establishing blood pressure model comprising: obtaining a plurality of first physiologic data of a plurality of general users and a plurality of first blood pressure data of the plurality of general users; performing a deep learning algorithm to establish a general blood pressure model according to the plurality of first physiologic data and the plurality of first blood pressure data, wherein the general blood pressure model has a parameter set and a loss function; obtaining a second physiologic data of a specific user and a second blood pressure data of the specific user; generating a blood pressure estimation according to the second physiologic data and the parameter set; calculating an error according to the blood pressure estimation, the second blood pressure data and the loss function; and adjusting the parameter set to establish a specific blood pressure model according to the error.

### BRIEF DESCRIPTION OF THE DRAWINGS

The present disclosure will become more fully understood from the detailed description given hereinbelow and the accompanying drawings which are given by way of illustration only and thus are not limitative of the present disclosure and wherein:

FIG. 1 is a system design diagram of establishing blood pressure models according to an embodiment of the present disclosure;

FIG. 2 is a flowchart of a method of establishing a blood pressure model according to an embodiment of the present disclosure;

FIG. 3 is a subsequent flowchart of a method of establishing a blood pressure model according to an embodiment of the present disclosure; and

FIG. 4 is a bar graph illustrating the accuracy of multiple blood pressure models established based on multiple first physiological data.

### DETAILED DESCRIPTION

In the following detailed description, for purposes of explanation, numerous specific details are set forth in order to provide a thorough understanding of the disclosed embodiments. It will be apparent, however, that one or more embodiments may be practiced without these specific details. In other instances, well-known structures and devices are schematically shown in order to simplify the drawings.

The method of establishing blood pressure model according to an embodiment of the present disclosure is suitable for a wearable and non-invasive blood pressure measurement device.

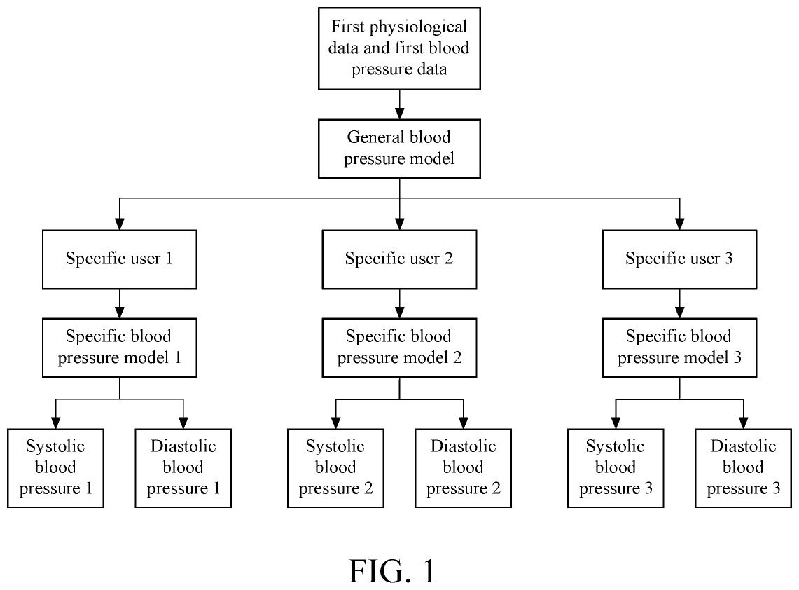

Please refer to FIG. 1. FIG. 1 is a system design diagram of establishing blood pressure models according to an embodiment of the present disclosure. The present disclosure collects a plurality of first physiologic data and a plurality of first blood pressure data from a group under test and uses these data and a deep learning neural network to generate a general blood pressure model. For each individual, namely, the specific user 1, the specific user 2 or the specific user 3, after obtaining a wearable blood pressure measurement device loaded with said general blood pressure model, the specific user may further adjust this general blood pressure model according to the user's physiologic data and blood data, thereby establishing a specific blood pressure model 1, a specific blood pressure model 2, or a specific blood pressure model 3 suitable for the specific user. After the specific blood pressure model 1 is established, the specific user 1 may use the wearable blood pressure measurement device loaded with the specific blood pressure model 1 to measure the user's systolic blood pressure and diastolic blood pressure. The above flow is also suitable for the specific user 2 and the specific user 3.

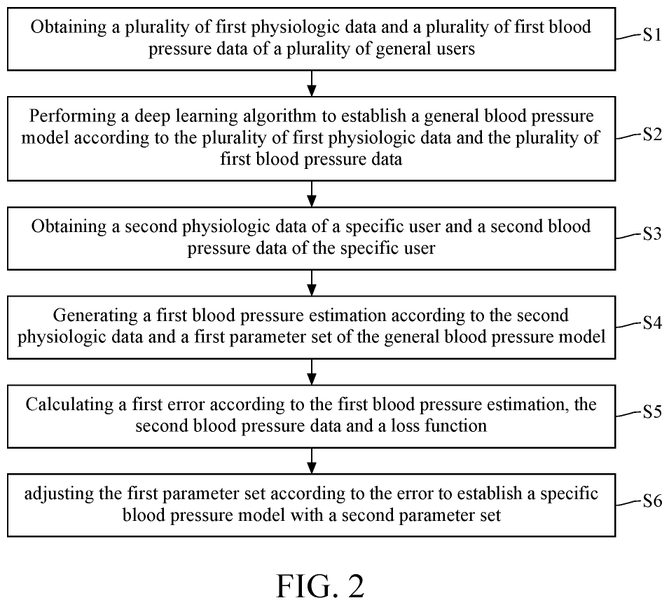

Please refer to FIG. 2. FIG. 2 is a flowchart of a method of establishing a blood pressure model according to an embodiment of the present disclosure.

Please refer to step S1, which shows “obtaining a plurality of first physiologic data and a plurality of first blood pressure data of a plurality of general users.” In an embodiment, the source of these first physiologic data and first blood pressure data is a public dataset. The public dataset is, for example, the University of Queensland vital signs dataset. However, the present disclosure does not limit to the above example. As long as the first physiological data and the first blood pressure data are obtained from a plurality of subjects, these data can be used as the data source of step S1. The type of the first physiologic data is an electrocardiography (ECG) signal, a photoplethysmography (PPG) signal, a synchronized signal including the above two signals, or a pulse transit time (PTT) calculated according to the ECG signal and the PPG signal. The first physiologic data includes a measurement value of systolic blood pressure and a measurement value of diastolic blood pressure. In an embodiment, the measurement value of systolic blood pressure and the measurement value of diastolic blood pressure are obtained by using a cuff-based sphygmomanometer to measure a general user. In an embodiment, the measurement to the same general user can be repeated multiple times. For example, the number of general users is 20. 15 ECG signals and 15 PPG signals are obtained from each of the general users and are served as the first physiological data, and 15 measurement values of systolic blood pressure and 15 measurement values of diastolic blood pressure are obtained from each of the general users and are served as the first blood pressure data.

According to the first physiological data obtained in step S1, a pre-processing procedure may be performed between step S1 and step S2. For example, the original signals of the first physiological data may be divided into 5-second segments, of which a linear filter such as the Savitzky-Golay filter is applied to. The segments with sharp edges or signal peaks will be selected, and the segments which do not have clear peaks or flags out (low variance) will be removed.

Please refer to step S2, which shows “performing a deep learning algorithm to establish a general blood pressure model according to the plurality of first physiologic data and the plurality of first blood pressure data”. In an embodiment, the type of each of the plurality of first physiologic data is an electrocardiography (ECG) signal, a photoplethysmography (PPG) signal, or a synchronized signal including the ECG signal and the PPG signal, and the deep learning algorithm is a convolutional neural network (CNN) which adopts multilayer perceptron (MLP) as a regressor. In other words, the general blood pressure model may be trained with ECG signals only, with PPG signals only, or with a timing synchronized signal containing ECG signals and PPG signals. In another embodiment, the type of each of the plurality of first physiologic data is a pulse transit time (PTT), which is an interval time between a peak of the ECG signals and a peak of the PPG signals, and the general blood pressure model is a linear regression.

After step S2 is finished, the general blood pressure model can be loaded in the blood pressure measurement device to perform a measurement to a specific user. In an embodiment, the measurement is performed directly based on the general blood pressure model. In another embodiment, steps S3-S6 in FIG. 2 will be performed to adjust the general blood pressure model into a specific blood pressure model according to the specific user's sensing data.

Please refer to step S3, which shows “obtaining a second physiologic data of a specific user and a second blood pressure data of the specific user”. In an embodiment, the second physiologic data of the specific user is measured by the sensors on the blood pressure measurement device. The type of the second physiologic data is the ECG signal, the PPG signal, a synchronized signal including the ECG signal and the PPG signal, or a PTT calculated by the ECG signal and the PPG signal. The second blood pressure data includes a measurement value of systolic blood pressure and a measurement value of diastolic blood pressure, the measurement value of systolic blood pressure and the measurement value of diastolic blood pressure are obtained by using a cuff-based sphygmomanometer to measure a specific user. In an embodiment, the step S3 can be performed repeatedly to obtain a plurality of second physiologic data and a plurality of second blood pressure data of the specific user. The second physiologic data and the second blood pressure data are used to adjust the general blood pressure model in the flow of steps S4-S6. The general blood pressure model can be adjusted into the specific blood pressure model which is more suitable for the specific user as long as more sensing data of the specific user are collected. In another embodiment, in addition to obtaining the second physiological data, a reference physiological data associated with the specific user may be further obtained in step S3. For example, the reference physiological data includes a specific user's body temperature when measuring the second physiological data, a cuff elasticity or a temperature when the specific user measures the second blood pressure data, or the height and weight of the specific user. These reference physiological data can be used to establish specific blood pressure models based on different scenarios. However, the present disclosure is not limited to the above examples.

Please refer to step S4, which shows “generating a first blood pressure estimation according to the second physiologic data and a first parameter set of the general blood pressure model”. The general blood pressure model has the first parameter set and the loss function, wherein said first parameter set is a collection of network weights when the general blood pressure model is trained by the neural network, or said first parameter set is a collection of parameters of every term of the linear function when the general blood pressure model adopts linear regression. In an embodiment, the first blood pressure estimation is obtained by inputting the second physiologic data into the general blood pressure model, and the value of the first blood pressure estimation can be a value of systolic blood pressure or a value of diastolic blood pressure, depending on the first blood pressure data used in training previously. In another embodiment, step S4 can be performed repeatedly to obtain a plurality of first blood pressure estimation if a plurality of second physiologic data of the specific user is obtained in step S3.

Please refer to step S5, which shows “calculating a first error according to the first blood pressure estimation, the second blood pressure data and a loss function”. In an embodiment, the calculation method of the first error is shown as follows.

L_{general}=∥BP− [symbol] ∥₂² (Equation 1)

wherein L_{general} is the loss function of the general blood pressure model; BP is the second blood pressure data of the specific user, such as the value of systolic blood pressure or the value of the diastolic blood pressure obtained by cuff-based sphygmomanometer; and [symbol] is the first blood pressure estimation.

Please refer to step S6, which shows “adjusting the first parameter set according to the first error to establish a specific blood pressure model with a second parameter set”. For example, if the general blood pressure model adopts a linear model, data points illustrated according to the second physiologic data and second blood pressure data may not fall on the curve corresponding to the linear model. Therefore, step S6 describes how to adaptively modify the curve corresponding to the linear model to minimize the error between the curve and the data points of the specific user. Step S6 performs a regularization procedure for learning the specific blood pressure model, as shown in Equation 2.

L_{calibration}=L_{general}+λ_{reg}L_{reg} (Equation 2)

wherein L_{calibration} is the loss function of the specific blood pressure model, L_{general} is the loss function of the general blood pressure model and λ_{reg} is the regularization parameter. The larger the value of λ_{reg} is, the higher the similarity degree between the general blood pressure model and the specific blood pressure model is. If the value of λ_{reg} is set to zero, the curve corresponding to the general blood pressure model will completely fit according to the data points of the specific user. L_{reg} is the regularization function of the regularization procedure, and the calculation method of L_{reg} is shown as Equation 3. In order to preserve the original characteristics of the general blood pressure model and to avoid the data points of the specific user completely dominating the loss function, L_{calibration} is adjusted according to L_{reg} and λ_{reg} whose value is set appropriately.

L_{reg}=∥η_{general}−η_{subject}∥₁¹ (Equation 3)

wherein η_{general} is the parameter set of the general blood pressure model, η_{subject} is the parameter set of the specific blood pressure model. In order to ensure that η_{subject} does not deviate too far from the originally learned parameters η_{general}, an embodiment of the present disclosure employs the L1-regularization to preserve the weight that contributes most to the blood pressure estimation.

According to the first error obtained in step S5, and the appropriately selected regularization parameter λ_{reg}, the loss function of specific blood pressure model may be optimized to establish a specific blood pressure model suitable for the specific user. The specific blood pressure model has a second parameter set, which is adjusted according to the first parameter set and the regularization procedure described in steps S5-S6.

In another embodiment, after step S5, “calculating the first error according to the first blood pressure estimation, the second blood pressure data and the loss function”, the present disclosure further comprises adjusting the parameter set to establish another specific blood pressure model according to a reference physiological data and the first error, wherein the reference physiological data includes a specific user's body temperature when measuring the second physiological data, or the height and weight of the specific user. In other words, the present disclosure may establish a plurality of specific blood pressure models of the specific user in different scenarios.

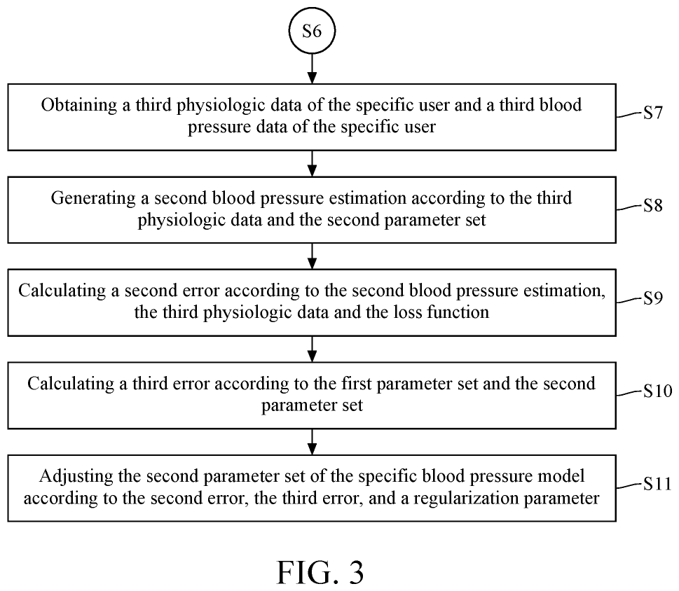

After finishing the flow of steps S1-S6 in FIG. 2, a specific blood pressure model suitable for the specific user is established. In order to further improve the measurement accuracy of the specific blood pressure model, the flow illustrated in FIG. 3 can be performed next. FIG. 3 is a subsequent flowchart of a method of establishing a blood pressure model according to an embodiment of the present disclosure.

Please refer to step S7, which shows “obtaining a third physiologic data of the specific user and a third blood pressure data of the specific user”. Basically, step S7 is identical to step S3. For example, the first data of the specific user is obtained in step S3, and the second data of the specific user is obtained in step S7.

Please refer to step S8, which shows “generating a second blood pressure estimation according to the third physiologic data and the second parameter set”. Basically, step S8 is identical to step S4, and the difference lies in that the second blood pressure estimation is calculated according to the second parameter set of the specific blood pressure model obtained in step S6, and the first blood pressure estimation described in step S4 is calculated according to the first parameter set of the general blood pressure model obtained in step S2.

Please refer to step S9, which shows calculating a second error according to the second blood pressure estimation, the third physiologic data and the loss function L_{general} shown in Equation 1. Basically, step S9 is identical to step S5.

Please refer to step S10, which shows “calculating a third error according to the first parameter set and the second parameter set”. In an embodiment, the third error is obtained by applying the first parameter set of the general blood pressure model and the second parameter set of the specific blood pressure model to the Equation 3. In other words, the third error is the calculation result of L_{reg}. However, the present disclosure does not limit the calculation method for the third error. For example, the third error can be calculated by mean squared error (MSE), mean absolute error (MAE), or cross entropy between the first parameter set and the second parameter set.

Please refer to step S11, which shows “adjusting the second parameter set of the specific blood pressure model according to the second error, the third error, and a regularization parameter) λ_{reg}”. Basically, Step S11 is identical to step S6.

Every time the flow shown in FIG. 3 is performed, the measurement accuracy of the specific blood pressure model is improved with new physiological data and blood pressure data of the specific user.

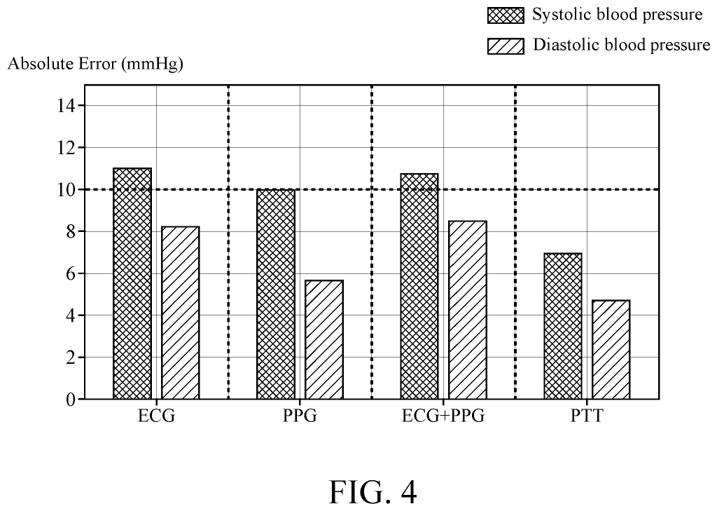

Please refer to FIG. 4. FIG. 4 is a bar graph illustrating the accuracy of multiple blood pressure models established based on multiple first physiological data. In FIG. 4, it shows that selecting PTT to train a general blood pressure model in step S1 can achieve that the error of systolic blood pressure is less than 7 mm Hg and the error of diastolic blood pressure error is less than 5 mm Hg. In the embodiment where a synchronization signal including the ECG signal and the PPG signal is used to train a general blood pressure model, the error of systolic pressure is less than 11 mm Hg, and the error of diastolic pressure error is less than 9 mm Hg. In general, using the PPG signal only to train a general blood pressure model or using the PTT to train a general blood pressure model can meet the blood pressure measurement standards followed by ANSI/AAMI/ISO in 2018, that is, the error is less than 10 mm Hg.

In view of the above, the present disclosure proposes a method of establishing blood pressure model and has the following effects: improving the accuracy of the cuffless blood pressure measurement, and providing a customized specific blood pressure model according to the physiological data of the specific user. The sphygmomanometer to which the present disclosure is applied do not interfere the user during a long-time measurement.

The present disclosure may establish the blood pressure model based on a variety of physiological signals, so it has more flexibility in choosing models.

What is claimed is:

1. A method of establishing blood pressure model comprising:
obtaining a plurality of first physiologic data of a plurality of general users and a plurality of first blood pressure data of the plurality of general users;
performing a deep learning algorithm to establish a general blood pressure model according to the plurality of first physiologic data and the plurality of first blood pressure data, wherein the general blood pressure model has a parameter set and a loss function;
obtaining a second physiologic data of a specific user and a second blood pressure data of the specific user;
generating a blood pressure estimation according to the second physiologic data and the parameter set;
calculating an error according to the blood pressure estimation, the second blood pressure data and the loss function; and
adjusting the parameter set to establish a specific blood pressure model according to the error.

2. The method of establishing blood pressure model of claim 1, wherein the parameter set of the general blood pressure model is a first parameter set, the blood pressure estimation is a first blood pressure estimation, the error is a first error, and the specific blood pressure model has a second parameter set;
after adjusting the parameter set to establish a specific blood pressure model according to the error, further comprising:
obtaining a third physiologic data of the specific user and a third blood pressure data of the specific user;
generating a second blood pressure estimation according to the third physiologic data and the second parameter set;
calculating a second error according to the second blood pressure estimation, the third physiologic data and the loss function;
calculating a third error according to the first parameter set and the second parameter set; and
adjusting the second parameter set of the specific blood pressure model according to the second error, the third error, and a regularization parameter.

3. The method of establishing blood pressure model of claim 2, wherein the third error is a mean squared error, a mean absolute error or a cross entropy of the first parameter set and the second parameter set.

4. The method of establishing blood pressure model of claim 1, wherein a type of each of the plurality of first physiologic data and the second physiologic data is an electrocardiography (ECG) signal, a photoplethysmography (PPG) signal, or a synchronized signal including the ECG signal and the PPG signal, and the deep learning algorithm is a convolutional neural network (CNN) which adopts multilayer perceptron (MLP) as a regressor.

5. The method of establishing blood pressure model of claim 1, wherein a type of each of the plurality of first physiologic data and the second physiologic data is a pulse transit time (PTT), and the general blood pressure model is a linear regression.

6. The method of establishing blood pressure model of claim 1, after calculating an error according to the blood pressure estimation, the second physiologic data and the loss function, further comprising: adjusting the parameter set to establish another specific blood pressure model according to a reference physiological data and the error.

7. The method of establishing blood pressure model of claim 1, before, further comprising:
dividing the plurality of first physiologic data into a plurality of segments; and
applying a linear filter on the plurality of segments.
